(19) United States
(12) Patent Application Publication
(10) Pub. No.: US 2009/0299224 A1
(43) Pub. Date: Dec. 3, 2009

(54) Lancet device and method for sampling and injecting blood using the lancet device

(76) Inventor: Jae Chern Yoo, Pohang-si, (KR)

Correspondence Address: STAAS & HALSEY LLP, SUITE 700, 1201 NEW YORK AVENUE, N.W., WASHINGTON, DC, 20005, US

(21) Appl. No.: 11/921,907

(22) PCT Filed: Jun. 8, 2006
(86) PCT No.: PCT/KR2006/002195

§ 371 Date: Jun. 23, 2009

(30) Foreign Application Priority Data
Jun. 8, 2005 (KR) 10-2005-0049571

(51) Int. Cl. A61B 5/151

(52) U.S. Cl. 600/583

(57) ABSTRACT

There is provided a lancet device including a lancet chamber and a blood outlet capable of drawing needed amount of blood from skin of mammals including humans by a vacuum mechanism or a capillary mechanism of the lancet device, temporarily storing the blood, and injecting the blood through an injection opening of a blood analysis and diagnosis apparatus such as a digital bio disc (DBD) and a lab-on-a-chip into a blood chamber thereof.

### CROSS-REFERENCE TO RELATED APPLICATIONS

This application claims the benefit under U. S. C. Section 371 of PCT Application No. PTC/KR2006/002195, filed Jun. 8, 2006, and Korean Application No. 10-2005-0049571, filed Jun. 8, 2006, in the Korean Intellectual Property Office, the disclosure of which is incorporated herein by reference.

### BACKGROUND

One or more aspect of embodiments relate to a blood sampling device, and more particularly, to a lancet device for sampling a small amount of blood required for a blood analysis and diagnosis apparatus such as a digital bio disc (DBD) and a lab-on-a-chip and a method for sampling and injecting the blood using the same. The lancet device and the method may be widely used for a disease test or a blood sugar test for diabetics.

A lancet device for puncturing skin of a person has been well known. For example, in order to sample a small amount of blood for a blood test, various types of lancet devices obtaining good results have been used in outpatient clinics of medical institutions, hospitals, the red cross hospitals, and emergency rescue organizations. A portion of the lancet device puncturing skin of a person has to be continuously maintained in an absolute aseptic condition. A portion of the lancet device for being injected into tissue to a predetermined depth to reach a blood vessel so as to draw blood from a wound is called a lancet. The lancet includes a steel needle having a circular cross section and a pointed end portion directed toward the skin. The lancet of the lancet device is disposable in order to avoid an infection problem. The lancet devices are disclosed in Deutsches Patent No. DE3111737 (Feb. 11, 1982) and U.S. Pat. No. 4,653,513 (Mar. 31, 1987). In such a lancet device, drops of blood are applied to a diagnostic reagent strip for disease or diabetic test through a blood outlet of the lancet device.

However, the lancet devices mainly have two problems. Firstly, it is difficult to draw an amount of blood as sufficiently and constantly as needed. Secondly, it is difficult to inject the blood drawn from the wound into a blood analysis and diagnosis apparatus such as a digital bio disc (DBD) and a lab-on-a-chip. A general blood analysis and diagnosis apparatus includes an injection opening through which the blood is injected and a blood chamber which stores the blood. However, when the blood in the wound is applied to the injection opening to inject the blood, most blood is applied to only the surrounding of the injection opening. Therefore, an amount of blood may be lost. Accordingly, in order to solve the second problem, the blood analysis and diagnosis apparatus needs to additionally include a blood collection device for collecting blood such as a capillary tube.

A vacuum lancet device is proposed in U.S. Pat. No. 6,152,942 and No. 4,895,147. The vacuum lancet device includes a lancet having a disposable needle and a holder into which the lancet is inserted to be held. When a user presses a handle of the vacuum lancet device, the needle punctures skin to draw blood by an elastic force of a spring included in the vacuum lancet device. In addition, a cap provided to an end portion of the vacuum lancet device is attached on the skin, and a sucking force generated by a vacuum mechanism is exerted so as to draw the blood.

However, the vacuum lancet device mainly has three problems. Firstly, when the blood is drawn out, the cap provided to the end portion of the vacuum lancet device is stained with the blood, so that the cap may be contaminated. Therefore, it is difficult to avoid loss of the drawn blood. Secondly, AIDS has become prevalent recently, so that use of needles may cause contagion of the disease. In addition, when a non-disposable cap provided to the end portion of the vacuum lancet device is contaminated by an infected person, the re-use of the vacuum lancet device may bring a fatal result. Thirdly, in order to collect the blood in the blood chamber, the blood analysis and diagnosis apparatus requires an additional blood collection device. Accordingly, there is needed a lancet device capable of avoiding blood-contamination of a cap provided to an end portion of a vacuum lancet device and directly injecting obtained blood into a blood analysis and diagnosis apparatus.

### SUMMARY

An aspect of the present invention provides a lancet device including a lancet chamber drawing a needed amount of blood from skin of mammals including humans by a vacuum mechanism or a capillary mechanism of the lancet device and a blood outlet for injecting the blood stored in the lancet chamber through an injection opening of a blood analysis and diagnosis apparatus such as a digital bio disc (DBD) and a lab-on-a-chip into a blood chamber thereof.

The present invention also provides a method for sampling and injecting blood required for a blood analysis and diagnosis apparatus such as a DBD and a lab-on-a-chip or a diagnostic reagent strip for disease or diabetic test by using the aforementioned lancet device.

According to an aspect of the present invention, there is provided a lancet device including a needle momentarily moving forward and restoring, the device including: a lancet cap having a lancet chamber or a capillary chamber enclosing the needle and sucking blood out-flowing from skin punctured by the needle to temporarily store the blood and a blood outlet draining the blood temporarily stored in the lancet chamber or the capillary chamber. More specifically, the lancet chamber or the capillary chamber may be a capillary tube having a cylindrical shape enclosing the needle for sucking the effluent blood from skin by a vacuum mechanism or a capillary mechanism to store the blood. In addition, the blood outlet may be formed at an end portion of the lancet chamber or the capillary chamber to drain the temporarily stored blood by an external pressure.

In the lancet device according to an aspect of the present invention, the blood outlet may serve as a blood sucking opening to suck the effluent blood into the lancet chamber or the capillary chamber. According to an aspect of the present invention, the term “vacuum” is used to include a negative pressure (a pressure under atmosphere). In addition, in the specification of the present invention, the terms “lancet chamber” and “capillary chamber” are used interchangeably.

In the above aspect, the lancet device including: a handle 1 driven by a user; a piston 2 connected with the handle 1 in a longitudinal direction and moving forward by pressing the handle 1; a cylinder 3 including a plunger 150 connected to the piston 2 to operate and a plurality of springs inducing momentary forward-moving and restoring (retracting) actions (trigger action) of the plunger; a needle 7 connected to an end portion of the plunger 150 and penetrating skin to a predetermined depth and restoring; a vacuum cap 4 connected to an end portion of the cylinder 3 and having an opening to be attached on the skin, thereby drawing the skin by a vacuum mechanism when the piston 2 is restored; and, the lancet cap 5 having the lancet chamber or the capillary chamber 8 enclosing the needle 7 and sucking the blood out-flowing from the punctured skin when the piston 2 is restored and temporarily storing the blood and the blood outlet 15 draining the blood temporarily stored in the lancet chamber or the capillary chamber by pressing the handle.

In the lancet device according to an aspect of the present invention, the piston 2 may further include a piston ring 10 attached on an inner surface of the cylinder 3 to hermetically seal the vacuum cap 4.

In the lancet device according to an aspect of the present invention, the vacuum cap 4 may include an exhaust port 4a and a flap element 4b automatically opening and closing the exhaust port 4a according to air pressure in the vacuum cap 4. In addition, the exhaust port 4a and the flap element 4b may be formed in the cylinder connected to the vacuum cap or a piston.

In the lancet device according to an aspect of the present invention, a plurality of the springs may include a third spring 91 momentarily moving forward the plunger by an expansive force and a second spring 89 restoring the momentarily moved plunger by a restoring force.

In the lancet device according to an aspect of the present invention, the end portion of the cylinder 3 may further include a vacuum ring so that the vacuum cap 4 is detachable from the cylinder.

In the lancet device according to an aspect of the present invention, the end portion of the cylinder 3 may further include a lancet ring 12 so that the lancet cap 4 is detachable from the cylinder.

In the lancet device according to an aspect of the present invention, the vacuum cap 4 and the lancet cap 5 may be made of a transparent plastic material.

In the lancet device according to an aspect of the present invention, the handle 1 may include a colored ring which is detachable therefrom.

In the lancet device according to an aspect of the present invention, the plunger 150 may include a plunger head 92a exerting a driving force during the trigger action and a lancet head 92b fixing the needle 7.

In the lancet device according to an aspect of the present invention, the plunger head 92a may hit a head portion of the lancet head 92b by a momentary forward-moving force during the trigger action to move the lancet head so as to inflict a wound on skin by the needle 7, and the lancet head may further include a fifth spring 68 for restoring the needle 7.

In the lancet device according to an aspect of the present invention, a head portion of the lancet head 92b may be provided with a ferromagnetic member or a magnet, an end portion of the plunger head 92a may be provided with a ferromagnetic member or a magnet having opposite polarity, so that the plunger head 92a is integrally coupled with the lancet head 92b by a magnetic attraction to exert the driving force during the trigger action.

In the lancet device according to an aspect of the present invention, a head portion of the lancet head 92b may be physically inserted into the plunger head 92a, so that the plunger head 92a is integrally coupled with the plunger head to exert the driving force during the trigger action.

In the lancet device according to an aspect of the present invention, the lancet device may further include a depth control means controlling a depth of the lancet inserted into the skin.

In the lancet device according to an aspect of the present invention, the depth control means may be formed by changing a position of a forward-movement preventing snag 51. In addition, the depth control means may be formed by controlling an interval between the lancet head 92b and the lancet chamber 8.

In the lancet device according to an aspect of the present invention, the lancet device may further include a gauge or level indication means for informing a user of a minimum amount of blood to be drawn on a surface of the lancet cap 5, the vacuum cap 4, or the lancet chamber or the capillary chamber 8.

In the lancet device according to an aspect of the present invention, the lancet chamber 8 may be a capillary tube having a cylindrical shape or a combination of a capillary tube having a cylindrical shape and a syringe needle. For example, when the capillary tube having a cylindrical shape is made of the same plastic as that of the lancet cap, the capillary tube may be coupled with a syringe needle. In this case, a diameter of the syringe needle or the capillary tube having a cylindrical shape may be larger than that of the needle 7 penetrating the skin to suck the blood by a vacuum mechanism or a capillary mechanism. The diameter of the syringe needle or the capillary tube may be in a range of from 0.05 mm to 4 mm.

According to another aspect of the present invention, there is provided a method of sampling and injecting blood using the aforementioned lancet device including: puncturing skin of a person using the lancet device to draw blood; storing temporarily the drawn blood in the lancet chamber or the capillary chamber by a vacuum mechanism or a capillary mechanism; fitting the blood outlet 15 into an injection opening 120 of a blood analysis and diagnosis apparatus; and transferring the blood temporarily stored in the lancet chamber or the capillary chamber through the blood outlet 15 into the blood analysis and diagnosis apparatus.

In the above aspect, the method may further include, after removing the vacuum cap 4 of the lancet device from the cylinder 3, fitting the blood outlet 15 into the injection opening 120 of the blood analysis and diagnosis apparatus, and transferring the temporarily stored blood to the blood analysis and diagnosis apparatus by pressing the handle.

According to still another aspect of the present invention, there is provided a method of sampling and injecting blood using the aforementioned lancet device including steps of: puncturing skin of a person using the lancet device to draw the blood; storing temporarily the drawn blood in a lancet chamber or a capillary chamber 8 by a vacuum mechanism or a capillary mechanism; and dropping the temporarily stored blood on a diagnostic reagent strip for disease or diabetic test through a blood outlet of the lancet device.

In addition, the method may further include dropping the temporarily stored blood on the diagnostic reagent strip for the diabetic test by pressing the handle 1 with or without removing the vacuum cap 4 of the lancet device from the cylinder 3.

### BRIEF DESCRIPTION OF THE DRAWINGS

These and/or other aspects and advantages will become apparent and more readily appreciated from the following description of the embodiments, taken in conjunction with the accompanying drawings of which:

FIG. 1 illustrates an outer appearance of a lancet device according to an embodiment.

FIG. 2A illustrates operations of a lancet device which is attached to skin to draw blood by a vacuum mechanism or a capillary mechanism, and FIG. 2B is a view showing operations of the lancet device which injects the drawn blood into a blood analysis and diagnosis apparatus or a digital bio disc (DBD) through an injection opening thereof according to an embodiment.

FIGS. 3A to 3D illustrate portions of a lancet device according to an embodiment.

FIG. 4 illustrates an assembled lancet device according to an embodiment of the present invention.

FIGS. 5A to 5C illustrate connection between a lancet head and a plunger head according to an aspect of the embodiment.

FIGS. 6A and 6B illustrate connection between a lancet head and a plunger head according to an aspect of the embodiment.

### DETAILED DESCRIPTION OF THE EMBODIMENTS

An aspect of the present invention provides a lancet device and a method of drawing needed amount of blood from skin by a vacuum mechanism or a capillary mechanism, temporarily storing the blood, and injecting the stored blood into a blood chamber of a blood analysis and diagnosis apparatus such as lab-on-a-chip through an injection opening thereof. Now, exemplary embodiments according to the present invention will be described in detail with reference to the accompanying drawings.

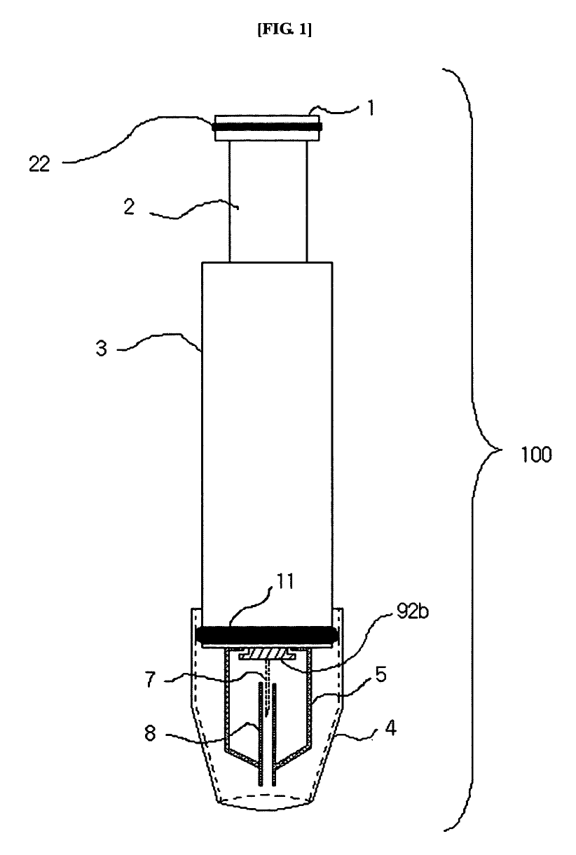

FIG. 1 is a side view showing an outer appearance of a lancet device 100 according to an embodiment. The lancet device 100 includes a handle 1 for being pressed to drive the lancet device 100, a piston 2 connected with the handle 1 in a longitudinal direction and pressed to move forward, a cylinder 3 including a plunger connected to the piston to operate and a spring supporting the plunger to perform momentary forward-moving and restoring actions (trigger action), a vacuum cap 4, and a lancet cap 5. The vacuum cap 4 and the lancet cap 5 are detachable from the cylinder 3. When the cylinder 3 is covered with the vacuum cap 4, the cylinder 3 is hermetically sealed with a vacuum ring 11. According to an embodiment, the vacuum ring 11 may be made of a rubber material. Reference numeral 22 denotes a colored ring which is detachable from the handle 1. Due to the color, users can distinguish the users' own lancet devices without confusing when a number of users use lancet devices having the same type but different color. According to an aspect of the present invention, the color ring 22 may have one of rainbow colors, for example. Therefore, the user may select one color and fit a ring having the color to the handle 1. In addition, the vacuum cap 4 and the lancet cap 5 according to an aspect of the present invention may be made of a transparent plastic such that the user may directly check sampling operations and sampling state. A needle 7 fixed to a lancet head 92b momentarily penetrates skin by a trigger action. Reference numeral 8 denotes a lancet chamber or a capillary chamber.

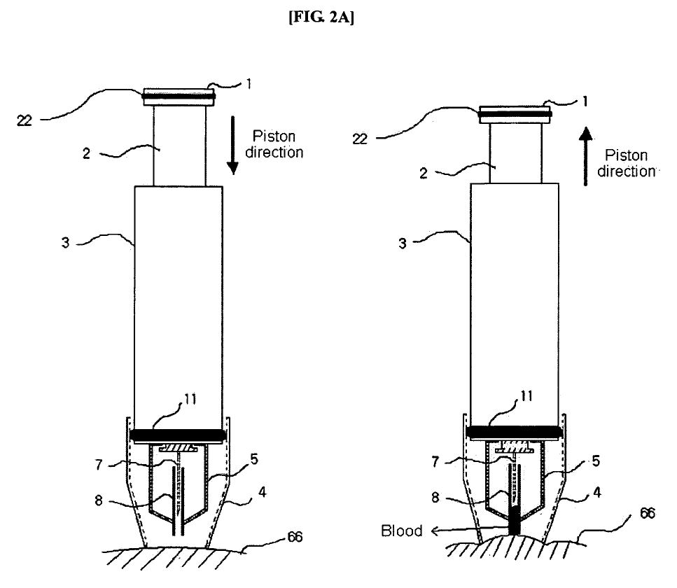

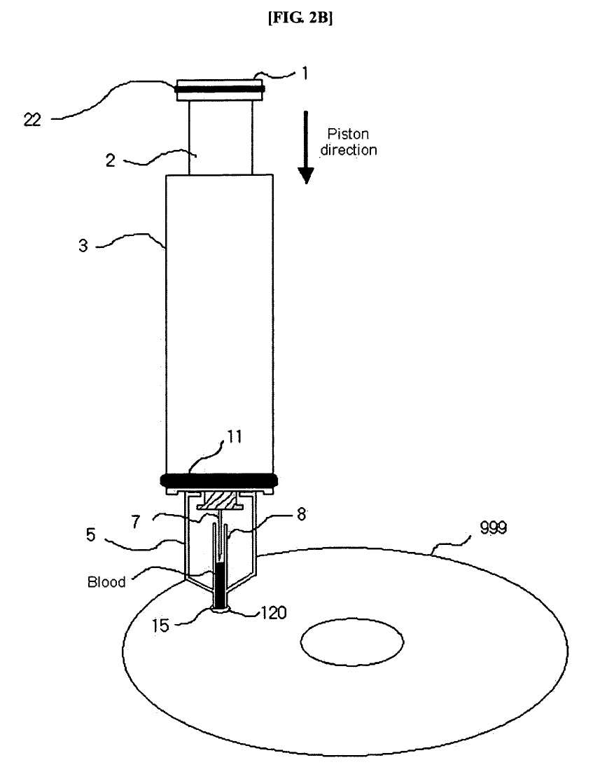

FIG. 2A is a view showing operations of the lancet device 100 which is attached to skin to draw blood by a vacuum mechanism or a capillary mechanism, and FIG. 2B is a view showing operations of the lancet device 100 which injects the drawn blood into a blood analysis and diagnosis apparatus or DBD through an injection opening thereof according to an embodiment of the present invention. The vacuum cap denoted by reference numeral 4 which is attached on the skin to block air intake, thereby maintaining the vacuum while the piston 2 is restored.

The left figure of FIG. 2A shows an example of an operation in that the handle 1 is pressed while the vacuum cap 4 of the lancet device 100 is attached on the skin 66 to sample blood.

In the right figure of FIG. 2A, according to the continuous pressure, a wound is inflicted on the skin by the trigger action, and the blood is drawn. In addition, since vacuum is generated by the restoring action of the piston 2, the skin is partially pulled into the vacuum cap 4, so that the skin is swollen. The blood drawn from the wound is temporarily stored in the lancet chamber 8, that is, a central conduit of the lancet cap 5.

FIG. 2B shows operations in that the blood temporarily stored in the lancet chamber 8 is transferred to the DBD or the blood analysis and diagnosis apparatus 999. After the vacuum cap 4 is removed from the cylinder 3, the blood outlet 15 is fitted into an injection opening 120, and the blood is transferred to the DBD or the blood analysis and diagnosis apparatus 999 by the press of the handle 1. More specifically, after the blood outlet 15 is closely fitted into the injection opening 120, the handle 1 is pressed, and the blood stored in the lancet chamber 8 is injected into the blood chamber of the blood analysis and diagnosis apparatus 999.

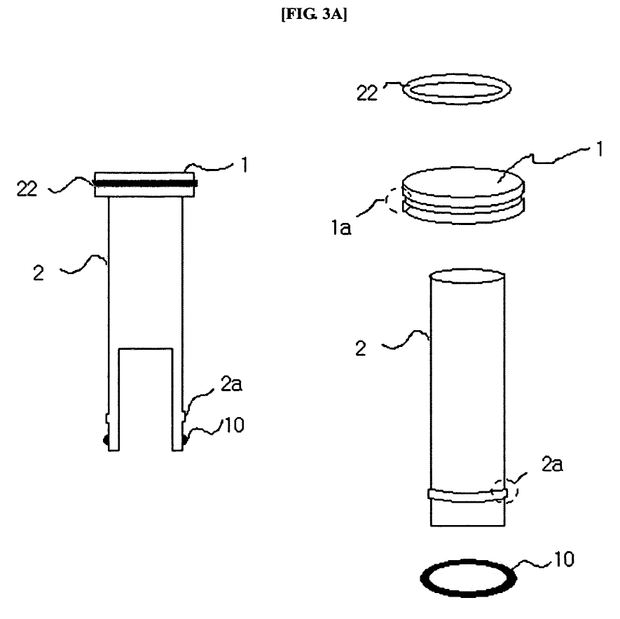

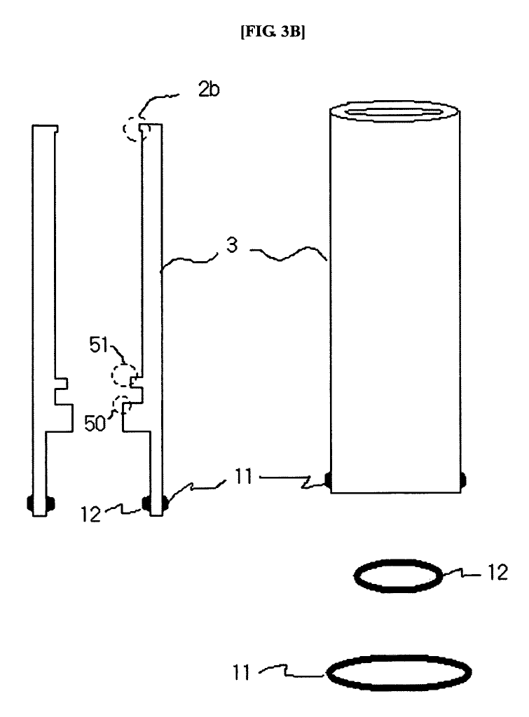

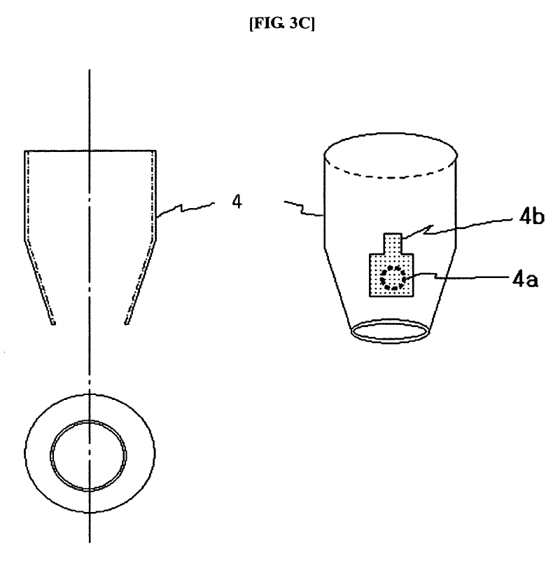

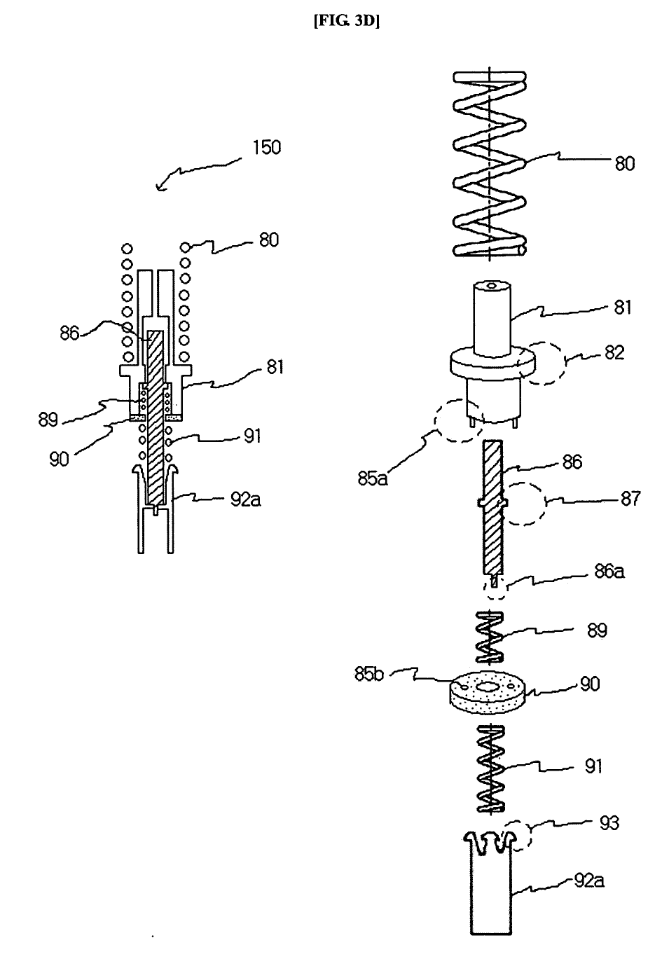

FIGS. 3A to 3D illustrate portions of the lancet device according to an embodiment of the present invention. FIG. 3A is an exploded view showing the handle 1 and the piston 2 and a sectional view showing an assembled state thereof. A piston ring 10 is fitted to the piston 2 to maintain vacuum by being attached to a cylinder wall when the piston 2 is restored. For option choices, the color ring 22 may be fitted to the handle 1 to distinguish the lancet device 100. The handle 1 is provided with a groove 1a for fitting the color ring 22, and a lower portion of the piston 2 is provided with a disengagement preventing snag 2a for preventing the piston from freely disengaging from the cylinder 3.

FIG. 3B illustrates the cylinder 3. Reference numeral 50 denotes a resisting snag, and reference numeral 51 denotes a forward-movement preventing snag. An end portion of the cylinder 3 is provided with the vacuum ring 11 and a lancet ring 12 to maintain the vacuum when the vacuum cap 4 and the lancet cap 5 are fitted to end portion of the cylinder 3. In addition, the cylinder 3 is provided with a cylinder snag 2b for cooperating with the disengagement preventing snag 2a to prevent the piston 2 from being disengaged from the cylinder 3.

FIG. 3C illustrates the vacuum cap 4. The cylinder 3 is sealed with the vacuum cap 4 by the vacuum ring 11. Reference numeral 4a denotes an exhaust port, and reference numeral 4b denotes a flap element for automatically opening and closing the exhaust port 4a according to air pressure in the vacuum cap 4. In the present invention, the flap element 4b may be made of a flexible plastic material or a rubber material. In the present invention, while the vacuum cap 4 is attached on the skin 66, the handle 1 is pressed, so that the flap element 4b is removed from a surface of the vacuum cap 4 to open the exhaust port 4a by an increase in pressure. Therefore the blood may be sampled. While the piston 2 performs the restoring action, air pressure in the vacuum cap 4 is dropped, so that the flap element 4b is attached to the surface of the vacuum cap 4 to close the exhaust port 4a, thereby generating vacuum.

FIG. 3D illustrates the plunger 150. The plunger 150 includes a plunger shaft 86, a plunger head 92a having a plurality of protrusions 93 protruding in a radial direction, and a plunger housing 81. In addition, the plunger 150 is assembled with a first spring 80, a second spring 89, and a third spring 91 to induce the trigger action. The plunger 150 also includes a plunger shaft snag 87 and a housing snag 90 to induce a compression of the second spring 89 while the plunger head 92a momentarily moves forward by the trigger action. Holes 85b of the housing snag 90 are assembled with housing pins 85a, and the housing snag 90 is coupled with the plunger housing 81 by thermal fusing. A shaft pin 86a is inserted into a hole provided to the plunger head 92a, and the shaft pin 86a is coupled with the plunger head 92a by thermal fusing.

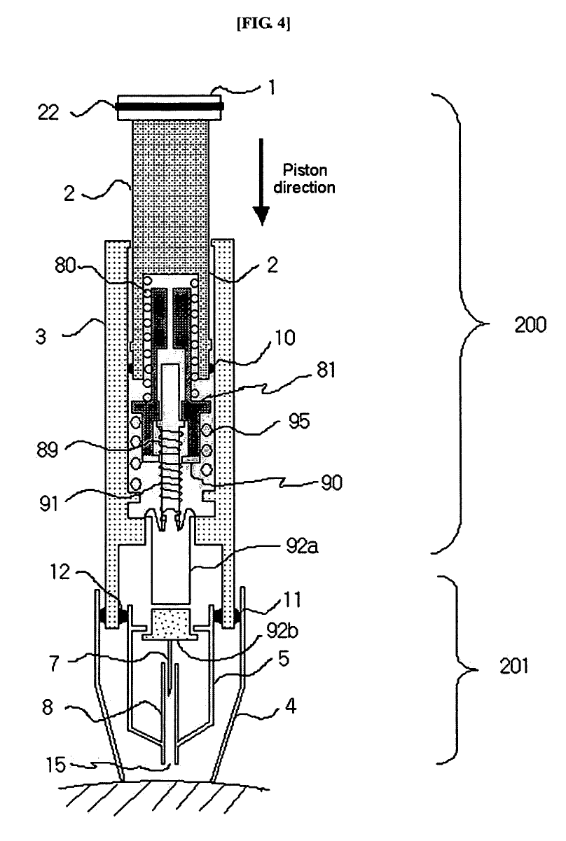

FIG. 4 illustrates an assembled lancet device 100 according to an embodiment of the present invention. The lancet device 100 mainly includes a handle portion 200 and a cap portion 201. The handle portion 200 includes the handle 1, the piston 2, and the cylinder 3. The cap portion 201 includes the vacuum cap 4 and the lancet cap 5. The handle portion 200 includes the handle 1 for being pressed by the thumb of the user and the piston 2 and the cylinder 3 for being grasped by the other fingers. The handle portion 200 and the cap portion 201 constitute the lancet device 100. The first to fourth springs 80, 89, 91, and 95 and the plunger 150 for inducing the trigger action by the press of the handle 1 are included in the cylinder 3. The lancet cap 5 includes the needle 7 having a pointed end portion for puncturing the skin and the lancet head 92b fixing the needle 7. The vacuum cap 4 and the lancet cap 5 are detachable from the cylinder 3. Before drawing blood, the lancet cap 5 is fitted to the lancet ring 12 of the cylinder 3, and the vacuum cap 4 is then fitted to the vacuum ring 11 of the cylinder 3.

The lancet cap 5 includes the lancet chamber 8 for temporarily storing the drawn blood and the blood outlet 15 for injecting the temporarily stored blood through the injection opening 120 of the blood analysis and diagnosis apparatus. In addition, when the blood is drawn, the blood outlet 15 serves as an opening to draw the blood by a vacuum mechanism or a capillary mechanism. The lancet cap 5 according to the present invention is disposable.

The plunger head 92a includes the plurality of protrusions 93 protruding in a radial direction. The protrusions 93 are caught by the resisting snag 50 provided to an inner wall of the cylinder 3 when the handle 1 is pressed.

In order to puncture the skin, the piston 2 is pushed by the handle 1 being pressed by a thumb of the user in an axial direction, so that the plunger housing 81 is also pushed in the axial direction. Here, the protrusions 93 are caught by the resisting snag 50, so that the resisting snag 50 is pressed, and the protrusions are elastically deformed to overcome the resistance formed by the press.

During the pressing operation, the third spring 91 is compressed. When the resistance is overcome by the continuous pressure, the protrusions 93 pass the resisting snag 50, and the plunger head 92a moves forward instantly, so that the needle 7 may be momentarily inserted into the skin of a person to a predetermined depth. The third spring 91 is compressed during the pressing operation.

In the present invention, a depth control means is formed by changing a position of the forward-movement preventing snag 51. More specifically, as the forward-movement preventing snag 51 is positioned closer to the handle 1, the plunger shaft 86 and the plunger head 92a cannot move forward that much, so that a depth of a portion of the needle inserted into the skin becomes shallower. On the contrary, as the forward-movement preventing snag 50 is positioned farther from the handle 1, the plunger shaft 86 and the plunger head 92a can move forward that much, so that the depth of the portion of the needle inserted into the skin becomes deeper.

The first and fourth springs 80 and 95 are drive springs for allowing the piston 2 to be pushed when a predetermined or more force is applied to the handle 1 in the axial direction to puncture the skin, and supporting the pushed second spring to restore. The second spring 89 and the third spring 91 are designed to allow the plunger 150 to perform the trigger action. More specifically, when the protrusions 93 are caught by the resisting snag 50 during pressing, the third spring 91 is compressed by the housing snag 90. In addition, when the protrusions 93 overcome the resisting snag 50, the third spring 91 momentarily expands and so provides an expansive force for momentarily moving the plunger head 92a. On the contrary, the second spring 89 supports the plunger head 92a momentarily moved by the expansion of the third spring to restore.

That is, after the protrusions 93 of the plunger head 92a pass the resisting snag 50 and are rapidly moved by the expansive force such that the needle 7 is inserted into the skin, the second spring 89 is used to instantly pull the needle 7 from the skin. At a moment when the plunger head 92a passes the resisting snag 50, the plunger head 92a rapidly moves forward, and at this time, the plunger housing 81 is stopped by the forward-movement preventing snag 51 and only the plunger shaft 86 and the plunger head 92a move forward. As a result, the second spring 89 is momentarily compressed by the plunger shaft 87 and the housing snag 90, and after that, the plunger head 92a is restored to the cylinder 3 by a restoring force for expanding the momentarily compressed second spring 89. Thereafter, the piston 2 is restored to release the pressure, so that vacuum is generated by the piston ring 10. Therefore, the skin is partially pulled into the vacuum cap 4, so that the blood is drawn from the wound into the lancet chamber 8. In this case, the blood is drawn into the lancet chamber 8 to be stored, so that the vacuum cap 4 is not contaminated by the blood.

An aspect of the present invention, an inflow of the blood into the lancet chamber 8 is caused by a vacuum mechanism or a capillary mechanism formed by the lancet device 100.

An aspect of the present invention, the lancet chamber 8 may be a syringe needle having a hole having a diameter ranging from 0.05 mm to 4 mm. In this case, the syringe needle may enclose the needle 7. Since the diameter of the syringe needle is very small, the capillary mechanism is easily performed. The needle 7 for penetrating the skin is disposed in the hole of the syringe needle. When the lancet device 100 is operated, the needle 7 performs momentary forward-moving and restoring actions. In addition, the lancet chamber 8 according to the present invention may be a combination of a capillary tube having a cylindrical shape and a syringe needle.

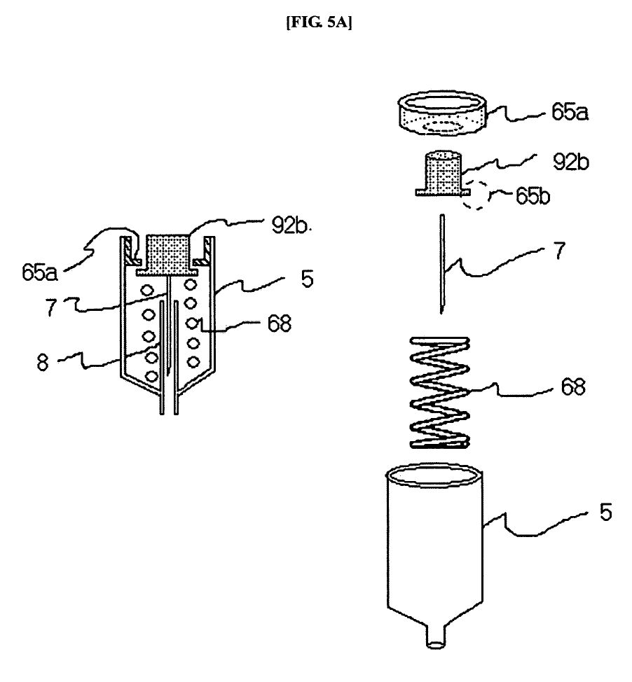

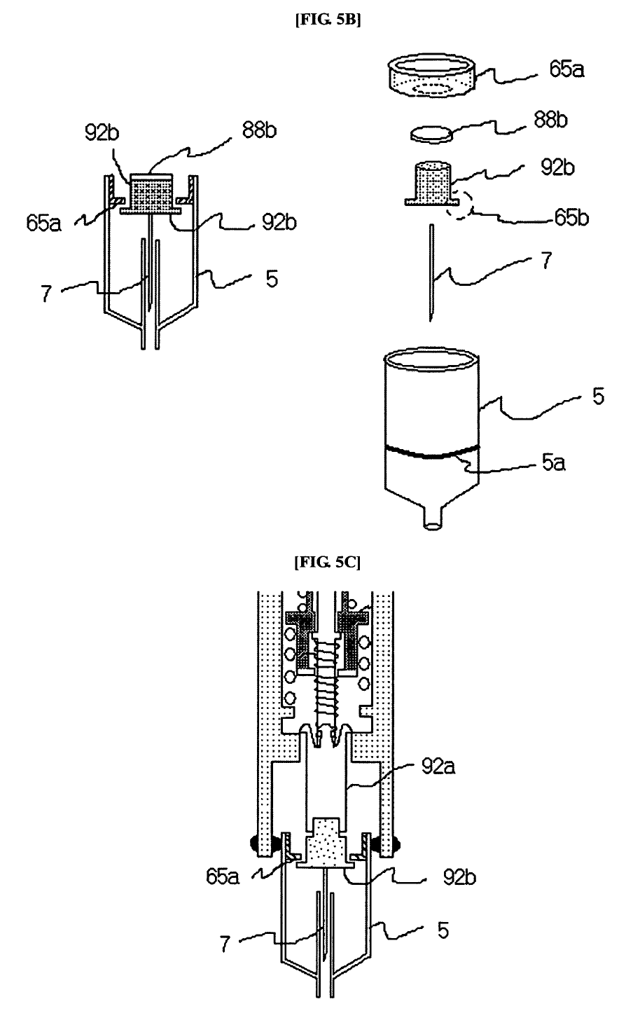

FIGS. 5A to 5C illustrate connection between a lancet head 92b and a plunger head 92a. The lancet cap 5 is provided with a lancet cap snag 65a and a head snag 65b to prevent the lancet head 92b from being freely removed from the lancet cap 5.

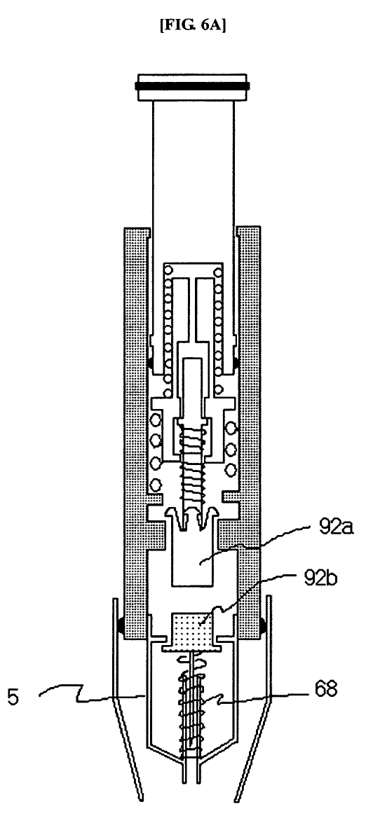

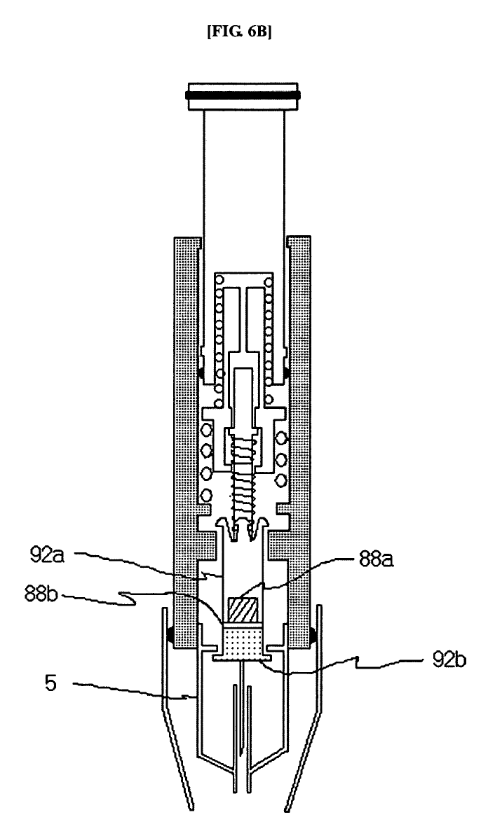

FIG. 5A illustrates an example of the lancet cap 5. The plunger head 92a hits a head portion of the lancet head 92b by a force from the momentary forward-moving action during the trigger action, so that the lancet head 92b is moved, and the needle 7 fixed to the lancet head 92b inflicts a wound on the skin. In this case, the lancet head 92b requires a fifth spring 68 to restore the needle 7. The left figure is a sectional view of the lancet cap 5, and the right figure is an exploded view thereof. FIG. 6A shows the lancet device 100 using the lancet head 92b and the plunger head 92a according to the example.

FIG. 5B illustrates an example of the lancet cap 5. A head portion of the lancet head 92b is provided with a ferromagnetic member or a magnet 88b, and an end portion of the plunger head 92a is also provided with a magnet 88a, so that the plunger head 92a is integrally coupled with the lancet head 92b by a magnetic attraction. In the trigger action, the plunger head 92a moves together with the lancet head 92b. The left figure is a sectional view of the lancet cap 5, and the right figure is an exploded view thereof. An aspect of the present invention, the magnets may be neodymium magnets. FIG. 6B shows a lancet device 100 using the lancet head 92b and the plunger head 92a according to the example. Reference numeral 5a represents a gauge or a level indication showing the user the minimum amount of blood to be sampled and shown by the surface of the vacuum cap 5.

FIG. 5C shows an example of the lancet cap 5. In the example, a head portion of the lancet head 92b is forcibly inserted into the plunger head 92a, so that the lancet head 92b is integrally coupled with the plunger head 92a.

While the present invention has been particularly shown and described with reference to exemplary embodiments thereof, it will be understood by those skilled in the art that various changes in form and details may be made therein without departing from the spirit and scope of the invention as defined by the appended claims. The exemplary embodiments should be considered in descriptive sense only and not for purposes of limitation. Therefore, the scope of the invention is defined not by the detailed description of the invention but by the appended claims, and all differences within the scope will be construed as being included in the present invention.

1. A lancet device including a needle momentarily moving forward and restoring, the device comprising:
a lancet cap having a vacuum lancet chamber or a capillary chamber enclosing the needle and sucking blood out-flowing from skin punctured by the needle to temporarily store the blood.

2. The device according to claim 1, further comprising:
a handle;
a piston connected with the handle in a longitudinal direction and moving forward by pressing the handle;
a cylinder including a plunger connected to the piston to operate and a plurality of springs inducing momentary forward-moving and restoring actions (trigger action) of the plunger;
a needle connected to an end portion of the plunger and penetrating skin to a predetermined depth and restoring;
a vacuum cap connected to an end portion of the cylinder and having an opening to be attached on the skin, thereby drawing the skin by a vacuum mechanism when the piston is restored; and,
the lancet cap having the lancet chamber or the capillary chamber enclosing the needle and sucking the blood out-flowing from the punctured skin when the piston is restored and temporarily storing the blood and the blood outlet draining the blood temporarily stored in the lancet chamber or the capillary chamber by pressing the handle.

3. The lancet device according to claim 2, wherein the piston comprises a piston ring attaching to an inner wall of the cylinder to hermetically seal the vacuum cap.

4. The lancet device according to claim 2, wherein the vacuum cap comprises an exhaust port and a flap element automatically opening and closing the exhaust port according to air pressure in the vacuum cap.

5. The lancet device according to claim 2, wherein a plurality of the springs comprise a third spring momentarily moving forward the plunger by an expansive force and a second spring restoring the momentarily moved plunger by a restoring force.

6. The lancet device according to claim 2, wherein the end portion of the cylinder comprises a vacuum ring so that the vacuum cap is detachable from the cylinder.

7. The lancet device according to claim 2, wherein the end portion of the cylinder further comprises a lancet ring so that the lancet cap is detachable from the cylinder.

8. The lancet device according to claim 2, wherein the vacuum cap and the lancet cap are made of a transparent plastic material.

9. The lancet device according to claim 2, wherein the handle comprises a colored ring which is detachable therefrom.

10. The lancet device according to claim 2, wherein the plunger comprises a plunger head exerting a driving force during the trigger action and a lancet head fixing the needle.

11. The lancet device according to claim 10,
wherein the plunger head hits a head portion of the lancet head by a momentary forward-moving force during the trigger action to move the lancet head so as to inflict a wound on skin by the needle, and
wherein the lancet head comprises a fifth spring for returning the needle.

12. The lancet device according to claim 10,
wherein a head portion of the lancet head is provided with a ferromagnetic member or a magnet,
wherein an end portion of the plunger is provided with a ferromagnetic member or a magnet having opposite polarity, and
wherein the plunger head is integrally coupled with the lancet head by a magnetic attraction to exert the driving force during the trigger action.

13. The lancet device according to claim 10, wherein a head portion of the lancet head is physically inserted into the plunger head, so that the plunger head is integrally coupled with the plunger head to exert the driving force during the trigger action.

14. The lancet device according to claim 2, further comprising a depth control means controlling a depth of the lancet inserted into the skin.

15. The lancet device according to claim 14, wherein the depth control means is formed by changing a position of a forward-movement preventing snag.

16. The lancet device according to claim 2, further comprising a gauge or level indication means for informing a user of a minimum amount of blood to be drawn on a surface of the lancet cap, the vacuum cap, or the lancet chamber or the capillary chamber.

17. The lancet device according to claim 1, wherein the lancet chamber is a capillary tube having a cylindrical shape or a combination of a capillary tube having a cylindrical shape and a syringe needle.

18. A method for sampling and injecting blood using the lancet device according to claim 1, the method comprising:
puncturing skin of a person using the lancet device to draw blood;
storing temporarily the drawn blood in the lancet chamber or the capillary chamber by a vacuum mechanism or a capillary mechanism;
fitting the blood outlet into an injection opening of a blood analysis and diagnosis apparatus; and
transferring the blood temporarily stored in the lancet chamber or the capillary chamber through the blood outlet into the blood analysis and diagnosis apparatus.

19. The method of claim 18, further comprising, after removing the vacuum cap of the lancet device from the cylinder, fitting the blood outlet into the injection opening of the blood analysis and diagnosis apparatus, and transferring the temporarily stored blood to the blood analysis and diagnosis apparatus by pressing the handle.

20. A method for sampling and injecting blood using the lancet device according to claim 1, the method comprising:
puncturing skin of a person using the lancet device to draw blood;
storing temporarily the drawn blood in the lancet chamber or the capillary chamber by a vacuum mechanism or a capillary mechanism; and
dropping the temporarily stored blood on a diagnostic reagent strip for disease or diabetic test through a blood outlet of the lancet device.

21. The method according to claim 20, further comprising dropping the temporarily stored blood on the diagnostic reagent strip for the diabetic test by pressing the handle with or without removing the vacuum cap of the lancet device from the cylinder.
